(19) United States
(12) Patent Application Publication
(10) Pub. No.: US 2006/0269493 A1
(43) Pub. Date: Nov. 30, 2006

(54) SILICATE AND OTHER OXIDE POWDERS WITH BONDED ANITMICROBIAL POLYMERS

(75) Inventor: William Toreki, Gainesville, FL (US)

Correspondence Address: ELMAN TECHNOLOGY LAW, P.C., P. O. BOX 209, SWARTHMORE, PA, 19081, US

(73) Assignee: QUICK-MED TECHNOLOGIES, INC., Gainesville, FL (US)

(21) Appl. No.: 11/462,884

(22) Filed: Aug. 7, 2006

Related U.S. Application Data
(63) Continuation-in-part of application No. PCT/US04/03272, filed on Feb. 5, 2004.

(51) Int. Cl. A61K 8/89; A61K 8/81; A61K 8/19

(52) U.S. Cl. 424/63; 424/70.12; 424/70.16

(57) ABSTRACT

This invention relates to methods and compositions for materials having a non-leaching coating that has antimicrobial properties. The coating is applied to substrates such as silicates, iron oxides, titanium dioxide, pigments, powders and other substrates. Covalent bonds are formed between the substrate and the polymer molecules that form the coating.

### CROSS-REFERENCE TO RELATED APPLICATIONS

This is a continuation-in-part of international patent application PCT/US04/03272, filed Feb. 5, 2004, published as WO 2005/084436A1.

### BACKGROUND OF THE INVENTION

It is known that certain quaternary ammonium salts possess antimicrobial properties. Examples include benzethonium chloride and benzalkonium chloride (BACTINE®). It is also known that certain low molecular weight quaternary ammonium groups can be incorporated into polymeric substrates (without chemical bonding) in order to provide certain degrees of antimicrobial activity.

Ionene polymers or polymeric quaternary ammonium compounds (polyquats), i.e., cationic polymers containing quaternary nitrogens in the polymer backbone, belong to a well-known class of biologically-active compounds. See, e.g., A. Rembaum, Biological Activity of Ionene Polymers, Applied Polymer Symposium No. 22, 299-317 (1973). Ionene polymers have a variety of uses in aqueous systems such as microbicides, bactericides, algicides, sanitizers, and disinfectants. U.S. Pat. Nos. 3,778,476, 3,874,870, 3,898,336, 3,931,319, 4,013,507, 4,027,020, 4,089,977, 4,111,679, 4,506,081, 4,581,058, 4,778,813, 4,970,211, 5,051,124, and 5,093,078 give various examples of these polymers, their preparation, and their uses. U.S. Pat. Nos. 3,778,476, 3,898,536, and 4,960,590, in particular, describe insoluble tri-halide containing ionene polymers. U.S. Pat. No. 4,013,507 describes ionene polymers which selectively inhibit the growth of malignant cells in vitro.

Hou et al., U.S. Pat. No. 4,791,063, teach polyionene-transformed modified polymer-polysaccharide separation matrices for use in removing contaminants of microorganism origin from biological liquids. This patent teaches that absorption of bacterial cells by ion-exchange resins is attributable to electrostatic attraction between quaternary ammonium groups on the resin surface and carboxyl groups on the bacteria cell surface.

The cerium (IV) ion initiated graft polymerization of vinyl monomers onto hydroxyl-containing substrates was first described by Mino (G. Mino and S. Kaizerman; “A New Method for the Preparation of Graft Copolymers. Polymerization Initiated by Ceric Ion Redox Systems”, Journal of Polymer Science 31(22), p 242 (1958)). The mechanism and kinetics of Ce(IV)-initiated graft polymerization of vinylacetate-acrylonitrile onto PVA in water solution was studied by Odian and Kho (G. Odian and J. H. T. Kho; “Ceric Ion Initiated Graft Polymerization onto Poly(vinyl Alcohol)”, J. Macromolecular Science Chemistry A4(2) p317-330, (1970)). Later, Vitta et al. described the grafting of methacrylic acid onto solid cellulose substrates (S.B. Vitta, et al., “The Preparation and Properties of Acrylic and Methacrylic Acid Grafted Cellulose Prepared by Ceric Ion Initiation. Part I. Preparation of the Grafted Cellulose”, J. Macromolecular Science—Chemistry A22(5-7) p 579-590 (1985)). None of these references describe antimicrobial materials, or graft polymers based on quaternary ammonium compounds.

### SUMMARY OF THE INVENTION

The present invention provides methods and compositions for an antimicrobial and/or antibacterial composition comprising a substrate over which a polymeric coating is non-leachably bonded. The polymeric coating contains a multitude of quaternary ammonium groups which exert activity against microbes, and also is absorptive of aqueous solutions. A method of fabrication is also described.

We have recently identified additional unexpected applications of the quaternary amine polymer chemistry defined herein. Such novel applications include the binding, whether through covalent linkages, ionic interactions, adsorption, or other mechanisms, of significant levels of quaternary amine polymers to powder substrates, including but not limited to mica. By the term “powder” for purposes of this invention, what is meant is monodisperse to polydisperse compositions of particle sizes ranging from the sub-micron (very fine) to millimeter size particles, depending on the relevant application. Mica is a commonly used component of cosmetics applied to the skin. Accordingly, application of this technology to the cosmetic arts is desirable to limit microbe proliferation and viability through use of antimicrobial powders. Additional applications of this technology include, for example, treatment of athlete's foot, (Tinia pedis), jock itch (Tinea cruris), chaffing, and other dermatological conditions in which opportunistic infections or irritations need to be controlled.

All patents, patent applications and publications discussed or cited herein are understood to be incorporated by reference to the same extent as if each individual publication or patent application was specifically and individually set forth in its entirety.

This invention provides significant levels of quaternary amine polymers bonded to powder substrates, including but not limited to mica, pigments, iron oxides, titanium oxides and the like for inclusion in cosmetic, antifungal, and like compositions whether in a dry or moist form.

This invention also provides compositions, including but not limited to cosmetics, in which commonly included components such as mica, kaolin, talc, pigments, titanium dioxide, iron oxides or the like are substituted with such oxides that have been treated with antimicrobially active quaternary amine polymers.

This invention also provides compositions in which cationic treated substrates in the form of a powder (including but not limited to micas),are modified through high level grafting of quaternary amine polymers to increase the capacity and avidity of dye molecule binding.

Furthermore, through high level grafting of quaternary amine polymers according to this disclosure, the properties of powders (including but not limited to micas) may be modified to increase the capacity and firmness of dye molecule binding to the cationic treated substrates of this invention.

It is an aspect of this invention to provide an inherently antimicrobial powder material comprised of an antimicrobial polymer bonded to the surface of a powder substrate in a non-leachable manner. Furthermore, it is an aspect of this invention that said antimicrobial polymer is a quaternary ammonium polymer. In one embodiment of this invention, the antimicrobial quaternary ammonium polymer is polyDADMAC, polyVBTAC, or polyTMMC. In another embodiment of this invention, the antimicrobial polymer is attached to the powder substrate via covalent chemical bonds using a coupling agent. In one embodiment, the coupling agent used is 3-(trimethoxysilyl)propyl methacrylate, an organosilane.

### DETAILED DESCRIPTION

Definitions:

For the purposes of this disclosure, certain definitions are provided. By “non-hydrolyzable” is meant a bond that does not hydrolyze under standard conditions to which a bond is expected to be exposed under normal usage of the material or surface having such bond. For instance, in a powder having an antimicrobial polymer bonded to its surface by “non-hydrolyzable” bonds according to the present invention, such “non-hydrolyzable” bonds do not hydrolyze (e.g., undergo a hydrolysis-type reaction that results in the fission of such bond) under normal storage conditions of such dressing, or exposure to wound exudates and/or body fluids when in use (e.g., under exposure to an expected range of pH, osmolality, exposure to microbes and their enzymes, and so forth, and added antiseptic salves, creams, ointments, etc.). The ranges of such standard conditions are known to those of ordinary skill in the art, and/or can be determined by routine testing.

By “non-leaching” is meant that sections of the polymer of the present invention do not appreciably separate from the material and enter a wound or otherwise become non-integral with the material under standard uses. By “not appreciably separate” is meant that no more than an insubstantial amount of material separates, for example less than one percent, preferably less than 0.1 percent, more preferably less than 0.01 percent, and even more preferably less than 0.001 percent of the total quantity of polymer. Alternately, depending on the application, “not appreciably separate” may mean that no adverse effect on wound healing or the health of an adjacent tissue of interest is measurable.

In regard to the above, it is noted that “non-leachable” refers to the bond between the polymer chain and the powder substrate. In certain embodiments of the present invention, a bond between the polymer backbone and one or more type of antimicrobial group may be intentionally made to be more susceptible to release, and therefore more leachable. This may provide a benefit where it is desirable for a percentage of the antimicrobial groups to be selectively released under certain conditions. However, it is noted that the typical bond between the polymer chain and antimicrobial groups envisioned and enabled herein are covalent bonds that do not leach under standard exposure conditions.

The term “quaternary ammonium” or “quaternary amine are used interchangeably. Both are common chemical nomenclature and their meaning will be understood by one skilled in the art.

By “degree of polymerization” is meant the number of monomers that are joined in a single polymer chain. For example, in a preferred embodiment of the invention, the average degree of polymerization is in the range of about 5 to 1,000. In another embodiment, the preferred average degree of polymerization is in the range of about 10 to 500, and in yet another embodiment, the preferred average degree of polymerization is in the range of about 10 to 100.

It is an aspect of this invention that a “coupling agent” is used. A coupling agent is a molecule used to form a bridging chemical bond between two different materials, such as between a metal oxide powder and an antimicrobial polymer. In general, coupling agents will posses dual chemical functionality. In other words, it will be capable of reacting by two different chemical mechanisms to link dissimilar materials. For instance the group of materials known as unsaturated alkoxy-silanes possesses siloxy-forming moieties which are capable of undergoing addition reactions with silicates and metal oxides. They also possess unsaturated moieties, such as vinyl groups, which are capable of undergoing addition polymerization with unsaturated monomers. A specific example is given below which illustrates the use of 3-(trimethoxysilyl)propyl methacrylate (an organosilane coupling agent) to join quaternary ammonium polymers to the surface of mica powder. An “organosilane” is a compound or molecule containing silicon atoms, wherein silicon atoms are covalently-bonded to one or more carbon atoms.

By “TMMC” is meant [2-(methacryloyloxy)ethyl]trimethylammonium chloride, a polymerizable vinyl monomer. By “polyTMMC” is meant a polymer comprised of TMMC. BY “DADMAC” is meant diallyldimethyl ammonium chloride, a polymerizable allyl monomer. By “polyDADMAC” is meant a polymer comprised of DADMAC. By “VBTAC” is meant vinylbenzyltrimethylammonium chloride, a polymerizable vinyl monomer. “By polyVBTAC” is meant a polymer comprised of VBTAC.

By the term “powder” for purposes of this invention, what is meant is monodisperse to polydisperse compositions of particle sizes ranging from the sub-micron (very fine) to millimeter size particles, depending on the relevant application.

Furthermore, it will be noted based on the present disclosure that antimicrobial applications of surface-treated mica have wide applicability to cosmetics, in which mica is an almost universally included component, with or without titanium dioxide treatment. Inclusion of mica treated according the present disclosure provides a solution, for example, to the situation where a mascara applicator is used, returned to a reservoir bearing adherent microbes which, in the absence of the antimicrobial mica, proliferate in the reservoir. Such proliferation has given rise to increasing levels of concern in the industry and this invention provides a novel, significant and unexpected solution to this long felt need. In addition, the increased dye-binding affinity of substrates, including mica, treated according to the present invention, has applicability to the fabric and cosmetic arts.

Antimicrobial” refers to the microbicidal or microbistatic properties of a compound, composition, article, polymer, powder, or material that enables it to kill, destroy, inactivate, or neutralize a microorganism; or to prevent or reduce the growth, ability to survive, or propagation of a microorganism.

“Microbe” or “microorganism” refers to any organism or combination of organisms such as bacteria, viruses, protozoa, yeasts, fungi, molds, or spores formed by any of these.

### EXAMPLE 1

Two grams of mica particles (<38 μm particle size) were placed into a solution of 0.1 g AMBP, 10 g of 65% DADMAC, and 10 g of water, then sparged with argon gas. The mixture was sealed in a jar under argon atmosphere and heated for 90 minutes at 80° C. The mixture was suspended in water (4 L), allowed to settle for several hours, and then re-suspended in fresh water. After settling overnight, the mica powder was washed several times in distilled water (50 mL aliquots), and washed by repeated shaking and centrifugation. The powder was then dried in a vacuum oven. Testing of the treated mica with a 1% solution of bromthymol blue dye produced a dark blue coloration after washing. Untreated mica powder tested in a similar manner showed no dye absorption. The powder was tested for antimicrobial activity against E. coli according to the method in the above example. Antimicrobial activity was high, (six log reduction) and no viable bacteria were observed.

### EXAMPLE 2

### Ionic Adsorption of polyDADMAC onto Mica Particles

A mixture of 5 mL water, 30 mL diallyldimethylammonium chloride monomer, “DADMAC” (65 wt % aqueous solution, Aldrich Chemical), and 0.2 g of 2,2′-azobis(2-methylpropionamidine)dihydrochloride, “V-50” (Aldrich Chemical) was sparged with argon gas, sealed in a glass jar under argon atmosphere, and heated overnight at 60° C. to prepare a solution of DADMAC homopolymer. Five grams of mica powder (<38 micron particle size) were added to the polymer solution, mixed well, and heated in an 80° C. oven for 1 hour. The mica powder was thoroughly washed 4 times with water. Approximately 100 mg of treated mica powder was mixed with 0.25 mL of bromthymol blue (BTB) dye solution (0.5% aqueous, pH kept at >7.5 by addition of dilute ammonia). The powder was allowed to settle, and excess dye solution was decanted. The powder was then washed three times with distilled water (3 mL each time). The powder remained blue in color; whereas, mica powder not treated with polyDADMAC retained no blue color after a similar dye treatment. It is presumed that the cationic sites on polyDADMAC displace some of the Na⁺ or K⁺ ions in the mica structure, and form ionic bonds with the negatively-charged polysilicate sites on mica. Without wishing to be bound by mechanism, it is presumed that only a fraction of the positively-charged quaternary nitrogen sites present on each polymer chain are complexed to the mica, with the remainder left free to react with, and bind to, the negatively-charged dye molecules. These same “free” quaternary nitrogen sites are available to provide the desired antimicrobial activity, while the chain, as a whole, remains tightly bonded to the surface of the mica particles. A sample of the DADMAC-treated mica was washed three times in a large excess of 5% NaCl solution, followed by washing three times in water. Subsequent dye treatment of the salt-washed mica revealed that most of the DADMAC polymer had been stripped from the mica surface, as evidenced by the observation of only a faint blue color. Since the poly-DADMAC may be leached from the thus-treated mica, applications in which slow release of antimicrobial would benefit from this composition.

### EXAMPLE 3

### In-Situ Polymerization of polyDADMAC onto Mica Particles

A solution was prepared and polymerized using the same formulation and conditions described in Example 2, except that the mica powder was added to the monomer solution before polymerization. The polymerized mixture was then dispersed in a large volume of water, and the mica washed several times as described in Example 2. Dye testing revealed similar results to those described in Example 2, except that the blue color was slightly darker, both before and after the mica was salt-washed. This indicates that in-situ polymerization leads to an increase of the surface concentration of quaternary groups in comparison to simply mixing the mica with polyquat. Without wishing to be bound by mechanism, possible explanations of this include the formation of some interpenetrating network structures between the mica and polyDADMAC, entrapment of polyDADMAC in pores of the mica, or covalent bonding between the polyDADMAC and the mica resulting from formation and reaction of radicals on the mica surface. Such radicals may be formed either from reaction with initiator species, or with growing polymer chains. It was observed that in polymerizations conducted according to the method of this Example that the polymerization reaction appeared to be accelerated by the presence of the mica powder. It was observed that the solutions became viscous faster, and at a lower temperature than similar solutions without mica. Once again, without wishing to be bound by mechanism, this phenomenon could be explained by a “template polymerization” effect wherein monomer adsorbed onto the mica surface is forced into a localized higher concentration resulting in a higher polymerization rate. Alternatively, the solvent (water) may diffuse into pores in the mica structure, thus giving an overall higher solution concentration of monomer. A third possibility is that the activation of the azo initiator is somehow facilitated by the mica surface.

### EXAMPLE 4

### Bonding of polyTMMC to Mica Particles

The experiments described in Example 2 and Example 3 (above) were repeated, except that DADMAC monomer was replaced by TMMC monomer [2-(methacryloyloxy)ethyl]trimethylammonium chloride (Aldrich Chemical). Reaction conditions were similar, except that the monomer concentration was lower (by approximately one-half) due to the higher reactivity of TMMC. The results obtained were similar to those in Examples 2 & 3, except that in all cases (adsorbed, in-situ, water-washed, and salt-washed) the colors of the TMMC-treated materials were somewhat darker than for the corresponding DADMAC-treated materials. A possible explanation is that the reaction conditions used produce TMMC polymers with a higher molecular weight than the DADMAC polymers. Nonetheless, the results achieved with different quaternary amine monomers indicate the generality of this approach to coating of mica particles.

### EXAMPLE 5

### In-Situ Polymerization of polyDADMAC onto Mica Particles Using a Coupling Agent

Ethanol (190 mL) was mixed with 10 mL distilled water. The pH of the solution was adjusted to approximately 4.5 using acetic acid. Twenty grams of a coupling agent: 3-(trimethoxysilyl)propyl methacrylate (Acros Organics) was added to the ethanol solution. The mixture was stirred for five minutes, and then 40 grams of mica powder with an average particle size of 16 microns was added to the mixture. The entire mixture was then stirred for approximately 18 hours in a sealed container. The mica was then allowed to settle and the supernatant liquid was carefully decanted. The mica powder was then washed twice with 100 mL of ethanol. The wet mica powder was allowed to partially dry in a fume hood at room temperature for one hour, then further dried in a vacuum oven at approximately 80° C. for 3 hours, then finally dried in air in a 120° C. oven. The powder was collected and lightly ground and sieved to break-up agglomerates. The treated powder was dispersed in a mixture of 50 mL diallyldimethylammonium chloride monomer, “DADMAC” (65 wt % aqueous solution, Aldrich Chemical), and 5 mL of a 10% aqueous solution of 2,2′-azobis(2-methylpropionamidine) dihydrochloride, “V-50” (Aldrich Chemical). The mixture was then thoroughly sparged with argon gas to remove dissolved oxygen, and sealed under argon atmosphere in a 900 mL glass jar. The sealed jar was then placed into an oven at 49° C. for approximately 18 hours. The jar was cooled and then hardened contents were thoroughly dispersed in approximately 800 mL of water with the aid of a mechanical blender. The mica was allowed to settle, and the supernatant liquid was decanted and discarded. The powder was then washed two additional times with water (approximately 200 mL each time), then washed three times with 5% NaCl solution (approximately 200 mL each), followed by four additional washings with water. Some of the washing steps utilized centrifugation in order to speed settling of the mica powder. The wet mica powder was then dried in an oven at 80° C. overnight. The dried powder was then collected and lightly ground to break-up agglomerates. Approximately 100 mg of treated mica powder was mixed with 0.25 mL of bromothymol blue (BTB) dye solution (0.5% aqueous, pH kept at >7.5 by addition of dilute ammonia). The powder was allowed to settle, and excess dye solution was decanted. The powder was then washed three times with distilled water (3 mL each time). The powder retained a dark blue color, indicating the presence of a high concentration of quaternary ammonium groups on the surface of the powder. The intensity of the blue color was significantly greater than the color of the material described in Example 2 (where coupling agent was not used), even before the material of Example 2 was washed with salt solution. This indicates that use of the coupling agent leads to an increased surface concentration of quaternary polymer, and that the quaternary polymer is covalently bonded to the mica particles, as it is not removed by washing with salt solution. As further proof of the stability of the bond between the mica and the quaternary polymer, a sample of the treated mica was heated in concentrated hydrochloric acid at approximately 80° C. for 5 minutes, and then was thoroughly washed with water. Treatment of the acid-washed material with BTB dye, as described above, again gave a dark blue product, thus indicating the stability of the polymer to mica bond.

### EXAMPLE 6

### Antimicrobial Activity of Mica with Surface-Bonded Quaternary Polymer

Samples of mica treated according to Example 5 were tumbled overnight in PBS solution containing a specified concentration of bacteria (˜1×10⁶/mL, a 10⁻² dilution of an overnight culture) with control samples having only bacteria with PBS. The mica was separated from the liquid in a centrifuge and aliquots of each sample were spread plated at a suitable dilution to determine the concentration of bacteria present after exposure to the sample. Sample efficacy was calculated as the difference between the sample and control population in units of log (CFU's). The general procedure is as follows:
1) 0.05 g mica sample was placed in a 15 mL, sterile, PP centrifuge tube.
2) Tube and sample were pasteurized in convection oven at 80° C. for 30 min.
3) 5 mL of sterile PBS was added to the tube.
4) 50 μL of an overnight culture of bacteria was added to the tube (ONC in 100% TSB)
5) The tube was tumbled at ˜45 rpm overnight (16 h) at room temperature (21° C.)
6) Serial dilutions of the solution in the tube were performed by placing 100 μL of solution into 9.9 mL of PBS as necessary to obtain discreet colonies when plated.
7) 100 μL of the diluted (or not) solutions was spread plated onto TSA plates and incubated at 37° C. overnight (16 h).
8) Colonies on the plates were counted and extrapolated to give the concentration of bacteria in the sample tubes.
9) The populations of the sample tubes were compared to the population in the control to determine the efficacy.

Experiments showed a reduction in population of 5 logs for S. Aureus, 7 logs for E. Coli, 2 logs for S. marcescens, and 4 logs for P. aeruginosa.

### EXAMPLE 7

### Covalent Bonding of Antimicrobial Quaternary Amine Polyers to Silicates Besides Mica

Following the procedures described above in EXAMPLES 2-6, silicates such as kaolin, talc and the like are similarly treated to confer antimicrobial efficacy to those compounds. Following the procedure of EXAMPLE 5, covalent bonding of polyquaternary ammonium compounds to talc, kaolin and the like is likewise achieved, with similar antimicrobial efficacy.

### EXAMPLE 8

### Bonding of Antimicrobial Quaternary Ammonium Polymers to Pigments, Including but Not Limited to Iron Oxides, or Other Metal Oxides, Such as Titanium Dioxide, and the Like

Following the procedures described above in EXAMPLES 2-7, bonding to iron oxides, other pigments, or titanium dioxide of polyquaternary ammonium compounds, such as poly-DADMAC, or poly-TMMC, is achieved. Again, use of an appropriate coupling agent, as in EXAMPLE 5, achieves covalent bonding of the polymer, and conference of antimicrobial efficacy to these products, which may be used in a wide variety of applications, including use in cosmetics, medicines and other compositions in which these compounds are typically included.

Having generally described this invention, including the best mode thereof, those skilled in the art will appreciate that the present invention contemplates the embodiments of this invention as defined in the following claims, and equivalents thereof. However, those skilled in the art will appreciate that the scope of this invention should be measured by the claims appended hereto, and not merely by the specific embodiments exemplified herein. Those skilled in the art will also appreciate that more sophisticated technological advances will likely appear subsequent to the filing of this document with the Patent Office. To the extent that these later developed improvements embody the operative principles at the heart of the present disclosure, those improvements are likewise considered to come within the ambit of the following claims.

What is claimed is:

1. A composition comprising a powder of a member of the group consisting of silicates, metal oxides, and cosmetic pigments having an antimicrobial polymer bonded to its surface via a coupling agent.

2. The composition of claim 1, wherein said antimicrobial polymer is non-leachably bonded.

3. The composition of claim 1 wherein said antimicrobial polymer contains quaternary ammonium moieties.

4. The composition of claim 3 wherein said antimicrobial polymer is polyDADMAC or polyTMMC.

5. The composition of claim 3 wherein said antimicrobial polymer is polyVBTAC.

6. The composition of claim 1, wherein said coupling agent comprises an organosilane.

7. The composition of claim 1, wherein said coupling agent is 3-(trimethoxysilyl)propyl methacrylate

8. The composition of claim 1, wherein said coupling agent is a derivative of 3-(trimethoxysilyl)propyl methacrylate.

9. A cosmetic composition comprising a powder of a member of the group consisting of mica, talc, kaolin, iron oxide, and titanium dioxide, the powder having an antimicrobial polymer bonded to its surface via a coupling agent.

10. The cosmetic composition of claim 9, wherein said antimicrobial polymer is non-leachably bonded.

11. The cosmetic composition of claim 9 wherein said antimicrobial polymer contains quaternary ammonium moieties.

12. The cosmetic composition of claim 11 wherein said antimicrobial polymer is polyDADMAC or polyTMMC.

13. The cosmetic composition of claim 11 wherein said antimicrobial polymer is polyVBTAC.

14. The cosmetic composition of claim 9, wherein said coupling agent comprises an organosilane.

15. The cosmetic composition according to claim 9, wherein said coupling agent is of 3-(trimethoxysilyl)propyl methacrylate.

16. The cosmetic composition according to claim 9, wherein said coupling agent is a derivative of 3-(trimethoxysilyl)propyl methacrylate.
